(19) United States
(12) Patent Application Publication
(10) Pub. No.: US 2006/0281735 A1
(43) Pub. Date: Dec. 14, 2006

(54) Antipsychotic combination therapies and compositions of an alpha-2 adrenergic receptor antagonist and an atypical antipsychotic neuroleptic

(76) Inventors: David Pickar, Chevy Chase, MD (US); Torgny Svensson, Lidingo (SE); Marie-Louise Wadenberg, Stockholm (SE)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/405,360

(22) Filed: Apr. 17, 2006

Related U.S. Application Data
(63) Continuation of application No. 10/522,699.
(60) Provisional application No. 60/398,718, filed on Jul. 29, 2002.
(60) Provisional application No. 60/398,719, filed on Jul. 29, 2002.
(60) Provisional application No. 60/398,720, filed on Jul. 29, 2002.
(60) Provisional application No. 60/402,542, filed on Aug. 12, 2002.
(60) Provisional application No. 60/433,781, filed on Dec. 17, 2002.
(60) Provisional application No. 60/433,782, filed on Dec. 17, 2002.
(60) Provisional application No. 60/433,785, filed on Dec. 17, 2002.

(51) Int. Cl. A61K 31/551; A61K 31/4178

(52) U.S. Cl. 514/220; 514/402

(57) ABSTRACT

The present invention provides novel antipsychotic therapies and compositions useful therein and provides methods for identifying new candidate molecules for the treatment of psychosis based on the proportional binding affinities for α₂ adrenergic and D₂ dopamine receptors.

This application is a Continuation of U.S. patent application No. Ser. 10/522,699, a national phase entry under 35 U.S.C. § 371 of International Application No. PCT/US03/13440, filed on Jul. 28, 2003, which claims the benefit of U.S. Provisional Patent Application Nos. 60/398,718, filed Jul. 29, 2002, 60/398,719, filed Jul. 29, 2002, 60/398,720, filed Jul. 29, 2002, 60/402,542, filed Aug. 12, 2002, 60/433,781, filed Dec. 17, 2002, 60/433,782, filed Dec. 17, 2002, and 60/433,785, filed Dec. 17, 2002. The entire contents of these applications are hereby incorporated by reference into the present specification.

### FIELD OF INVENTION

The present invention relates to improved antipsychotic therapeutic regimens that enable dose reduction and reduce the threshold of D₂ dopamine receptor occupancy required for an effective antipsychotic response and compositions for use therein. In particular, the present invention relates to therapies that involve the administration of a α₂ adrenergic receptor antagonist and an antagonist of D₂ dopamine and the 5HT₂receptors to produce an improved treatment for serious psychotic mental illness.

### BACKGROUND OF INVENTION

All patents and publications cited herein are incorporated by reference in their entirety. Full citations are provided at the end of the specification.

Conventional antipsychotics, also termed “typical antipsychotics” are effective in improving symptoms of schizophrenia and are characterized by their antagonist affinity for the D₂ dopamine receptor (see Creese et al, 1979 and Seeman et al, 1976). This pharmacological effect results in acutely diminished activity of the brain's dopamine neurotransmitter systems (Snyder et al 1976). Conventional antipsychotics can be classified into high, medium and low potency based on their proportional affinity for the D₂ receptor. Conventional or “typical” antipsychotics include chlorpromazine, fluphenazine, haloperidol, loxapine, mesoridazine, molindone perphenazine, pimozide, thioridazine, thioxthixene, trifluoperidone (The Merck Manual of Diagnosis and Therapy, 17^{th} Edition, pp 1563-1573, 1999). Significant numbers of patients suffering from schizophrenia and other psychoses have proven resistant to treatment with conventional antipsychotics. Moreover, conventional antipsychotics produce movement related adverse effects related to disturbances in the nigrostriatal dopamine system. These extrapyramidal side effects (EPS) include Parkinsonism, akathisia, tardive dyskinesia and dystonia. See Baldessarini and Tarazi in Goodman & Gilman's The Pharmacological Basis of Therapeutics 10^{th} Edition, 2001, pp 485-520.

“Atypical” antipsychotics refer to antipsychotic drugs that produce antipsychotic effects with little or no EPS and include clozapine, risperidone, olanzapine, quetiapine, ziprasidone and aripiprazole. “Atypical” antipsychotics differ from conventional antipsychotics in their pharmacological profiles. While conventional antipsychotics are characterized principally by D₂ dopamine receptor blockade, atypical antipsychotics show antagonist effects on multiple receptors including the 5HT₂ₐ and 5HT_{2c} serotonin receptors and varying degrees of receptor affinities. See Meltzer in Neuropsychopharmacology: The Fifth Generation of Progress, 2002, pp 819-831; Meltzer et al. 1989; and Baldessarini and Tarazi in Goodman & Gilman's The Pharmacological Basis of Therapeutics 10^{th} Edition, 2001, pp 485-520. Atypical antipsychotic drugs are commonly referred to as serotonin/dopamine antagonists, reflecting the influential hypothesis that greater affinity for the 5HT₂ receptor than for the D₂ receptor underlies “atypical” antipsychotic drug action or “second generation” antipsychotic drugs (Meltzer et al, 1989).

The most precise distinction between typical and atypical antipsychotics is the dissociation of conditioned avoidance response (CAR), an animal model of psychosis, from catalepsy (CAT), the animal model of EPS. While conventional antipsychotic drugs produce CAT and suppression of CAR at roughly equivalent doses, atypical antipsychotics suppress CAR at doses at least 2 fold lower than doses that produce CAT. See See Meltzer in Neuropsychopharmacology: The Fifth Generation of Progress, 2002, pp 819-831); Arnt and Skarsfeld, 1998). It has been shown that CAT and CAR models have high predictive value because they share occupancy of the D₂ receptor as an underlying mechanism. Dissociation of CAT from CAR translates into a clinical profile of an antipsychotic drug that produces EPS at doses that produce ≧80% in vivo D₂ occupancy with antipsychotic effects ≦70% D₂ occupancy. See Wadenberg et al., 2000, 2001.

Clozapine is the only antipsychotic drug with proven efficacy in treatment-refractory schizophrenia, with efficacy rates in treatment-refractory patients ranging from 20% to 70% and a therapeutic profile that includes reduction in suicide risk (See Kane et al, 1988; Pickar et al 1992; Pickar et al 2003; Miyamoto et al, 2001; Chakos et al, 2001; Meltzer et al, 2003). Unlike other atypical antipsychotics, clozapine is an effective antipsychotic with in vivo D₂ occupancy in the range of 50-60% considerably less than olanzapine (70%) and risperidone (≧70%) See Farde et al, 1992; Nyberg et al, 1993, Pickar et al 1996; Su et al 1997; Nordstrom et al 1998; Kapur et al 1999. High levels of D₂ occupancy (≧70%) are associated with EPS, dysphoria and poor subjective experience (see Kapur et al 2000 and Haan et al 2003). Antipsychotic potency with low in vivo D₂ occupancy is a highly desirable model for future antipsychotic drugs (See Kapur and Seeman, 1999). Consistent with its clinical profile, clozapine demonstrates pronounced separation of CAT from suppression of CAR in animal models (See Waddenberg et al 2000 and 2001).

Pharmacologically, clozapine differs from other atypical antipsychotics by antagonist affinity for the α₂-adrenergic receptor that exceeds its affinity for the D₂ receptor. Thus, while clozapine demonstrates about 1-4 fold greater affinity for α₂ receptor than D₂ receptor other atypical agents show markedly less affinity for the α₂ adrenergic receptor than for the dopamine D₂ receptor. For example, risperidone: 2-16 fold less, olanzapine: 8-21 fold less, quetiapine: 6-16 fold less, ziprasidone: 100 fold less, and aripiprazole: 23 fold less. See Baldessarini and Tarazi in Goodman & Gilman's The Pharmacological Basis of Therapeutics 10^{th} Edition, 2001, p 495; Miyomota in Neuropsychopharmacology: The Fifth Generation of Progress, 2002, p. 778; Shapiro et al, 2003. It was previously disclosed in two patents by Pickar et al. that serious psychotic disorders can be effectively treated using a combination of an α₂ adrenergic receptor antagonist and a D₂ dopamine receptor antagonist (see U.S. Pat. No. 5,492,907 and U.S. Pat. No. 5,663,167; Litman et al 1996; Hertel et al. 1999. Further, Broekkamp et al, U.S. Pat. No. 6,150,353, disclose combinations of the antidepressant mirtazapine with typical and atypical antipsychotic agents for the treatment of psychotic disorders.

The use of clozapine, however, is associated with severe side effects, including agranulocytosis, seizures, weight gain and diabetes. Weight gain and increased diabetes risk are adverse effects of olanzapine, while increased prolactin secretion is an adverse effect of risperidone.

Therefore, it is an object of the present invention to provide an effective method of treatment for patients suffering from serious psychotic mental illness who remain symptomatic despite treatment with drugs that block the D₂ dopamine and 5HT₂ serotonin receptors.

It is another object of the present invention to provide a method of treatment which does not have the severe side effects associated with the administration of clozapine.

It is a further object of the present invention to provide a means to minimize adverse events of atypical antipsychotics by enabling dose reduction and antipsychotic effectiveness at ≦70% in vivo D₂ occupancy.

It is yet another object of the present invention to provide pharmaceutical compositions and means for discovery of further compounds useful in foregoing methods of treatment.

Other objects, features and advantages of the present invention will become apparent to those skilled in the art from the following detailed description. It is to be understood, however, that the detailed description and specific examples, while indicating preferred embodiments of the present invention, are given by way of illustration and not limitation. Many changes and modifications within the scope of the present invention may be made without departing from the spirit thereof, and the invention includes such modifications.

### SUMMARY OF THE INVENTION

The present invention provides a method for treating a serious psychotic mental illness comprising the step of administering to a patient in need of such treatment a therapeutically effective amount of a combination of (i) an α₂ -adrenergic receptor antagonist and (ii) an atypical antipsychotic which has a greater antagonist affinity for D₂ dopamine receptor than its antagonist affinity for α₂ adrenergic receptor in a pharmaceutically acceptable carrier.

The invention further provides a pharmaceutical composition comprising a combination of (i) an α₂-adrenergic receptor antagonist, (ii) an atypical antipsychotic which has a greater antagonist affinity for D₂ dopamine receptor than its antagonist affinity for α₂ adrenergic receptor, and (iii) a pharmaceutically acceptable carrier, wherein the amount of said ingredients (i) and (ii) is therapeutically effective against serious psychotic mental illness.

The invention also provides a method for treating a serious psychotic mental illness comprising the step of administered to a patient in need of such treatment a therapeutically effective amount of a combination of (i) a compound having combined D₂ dopamine and 5HT₂ serotonin antagonist activities, wherein said compound (ii) has a greater antagonist affinity for D₂ dopamine receptor than its antagonist affinity for α₂ adrenergic receptor and (ii) a compound having α₂ adrenergic receptor antagonist activity.

In one embodiment, the invention provides a method for treating a serious psychotic mental illness comprising the step of administering to a patient in need of such treatment a therapeutically effective amount of a combination of idazoxan and olanzapine.

The invention also provides a method for treating a serious psychotic mental illness in a patient in need thereof which comprises co-administration of (i) a compound having combined D₂ dopamine and 5HT₂ serotonin antagonist activities, wherein said compound has a greater antagonist affinity for D₂ dopamine receptor than its antagonist affinity for α₂ adrenergic receptor, and (ii) a compound having α₂ adrenergic receptor antagonist activity, wherein said compound (i) is administered initially alone in an amount and for a period of time sufficient to stabilize said patient and subsequently said compound (ii) is co-administered in an amount and for a period of time that allows for a reduction in the amount of compound (i) administered to said patient.

The invention also provides a method for treating a serious psychotic mental illness comprising the step of administered to a patient in need of such treatment a therapeutically effective amount of a combination of (i) a compound that blocks or down-regulates D₂ dopamine and 5HT₂ serotonin antagonist activities and (ii) a compound that blocks or down-regulates α₂ adrenergic receptor activity.

The invention further provides a method for treating a serious psychotic disorder in a patient in need thereof which comprises administering an atypical antipsychotic in combination with an effective amount of an α₂ antagonist to provide antipsychotic effects at D₂ receptor occupancy levels of less than or equal to 60%.

The invention also provides a method for treating a serious psychotic disorder in a patient in need thereof which comprises administering an atypical D₂ antagonist in combination with an effective amount of a compound which enhances noradrenergic synaptic activity to provide antipsychotic effects at D₂ receptor occupancy levels of less than or equal to 60%.

The invention further provides a method for treating a serious psychotic illness comprising administering at least one atypical antipsychotic and at least one α₂ adrenergic receptor antagonist wherein the dosage balance between the atypical antipsychotic and α₂ antagonist is equivalent to a ratio of 900-1100 mg equivalents of chlorpromazine and an amount of an α₂ antagonist that provides for about equal D₂/α₂ receptor saturation.

The invention also provides a method for identifying compounds that are useful to treat serious psychotic mental illness which comprises subjecting a candidate compound to an assay demonstrating affinity for the D₂ dopamine receptor and an assay demonstrating affinity for the α₂ adrenergic receptor and determining that the compound demonstrates significant affinity for both the D₂ dopamine receptor and the α₂ adrenergic receptor.

### BRIEF DESCRIPTION OF THE FIGURE

FIG. 1 depicts the effect of olanzapine (1.25 or 2.5 mg/kg, i.p.) and idazoxan (1.5 mg/kg s.c.) alone and in combination on Conditioned Avoidance Response (CAR) behavior in 9 rats serving as their own controls in a change-over design (Li, 1964). Shown are medians±semi-interquartile range. Statistics are shown as *p<0.05, **p<0.01 (compared to vehicle controls) and ++p<0.01 (compared to animals treated with olanzapine alone) as assessed by the Wilcoxon matched-pairs signed-ranks test. Idazoxan (1.5 mg/kg s.c.) alone had no effects on CAR performance. No escape failures were noted at any time or in any treatment group.

### DETAILED DESCRIPTION

It has been discovered that the administration of an α₂ adrenergic receptor antagonist unexpectedly enhances the antipsychotic potency of atypical antipsychotics. This is reflected by an enhanced dissociation of the dose of atypical antipsychotic required to produce CAT and the dose required to suppress CAR, animal models of EPS and antipsychotic potency, respectively. While atypical antipsychotics such as olanzapine are characterized by some separation of doses required for CAT and CAR, the addition of an α₂ antagonist substantively enhances this effect resulting in a more potent antipsychotic composition without enhancement of EPS liability. The present invention provides for an improved treatment for patients suffering from serious psychotic mental illness who remain symptomatic despite treatment with atypical antipsychotic drugs.

Methods of Treatment

As discussed above, it was previously reported by the present inventor that the administration of an α₂-adrenergic receptor antagonist enhances the therapeutic effect of conventional or “typical” antipsychotic drugs. Particularly, as is the subject of U.S. Pat. No. 5,492,907 and U.S. Pat. No. 5,663,167 by Pickar et al. it was discovered that the combined administration of an α₂ adrenergic receptor antagonist and a typical or conventional antipsychotic neuroleptic is an effective improvement upon monotherapy with a typical antipsychotic in patients suffering from serious psychotic mental illness. By contrast, the present invention in part involves the discovery that serious psychotic illnesses can be improved by the administration of an atypical antipsychotic drug and an α₂ adrenergic receptor antagonist. Further, the invention discovers that the addition of an α₂ antagonist to an atypical antipsychotic, by enhancing the separation between EPS liability and antipsychotic effectiveness, enables dose reduction of the atypical antipsychotic drug such that therapeutic effects can be gained with an in vivo D₂ occupancy ≦60%. On the basis of these discoveries, the invention provides for a model for high throughput screening of drug molecules to identify agents with enhanced antipsychotic potency.

“Atypical” antipsychotic drugs are distinguished from “typical” or conventional antipsychotic drugs on the basis of the dissociation of EPS liability from antipsychotic effectiveness as demonstrated in animal models (e.g., CAT and CAR) and in the clinic. Pharmacologically, “atypical” antipsychotic drugs differ prominently from conventional antipsychotics by displaying diverse pharmacological effects on multiple neurotransmitter systems, characteristically antagonist properties to both the D₂ and D₃ dopamine receptors and the 5HT₂ₐ and 5HT_{2c} serotonin receptors and include olanzapine, quetiapine, ziprasidone, aripiprazole, sertindole and risperidone. In the present invention, “atypical” antipsychotic drugs exclude, clozapine, an antipsychotic with a unique therapeutic profile and with antagonist affinity for the α₂ adrenergic receptor that equals or exceeds the affinity for the D₂ dopamine receptor. Clozapine is associated with severe side effects including agranulocytosis, seizures and diabetes.

Examples of “typical” antipsychotic drugs include those referred to as “typical neuroleptics” such as chlorpromazine, fluphenazine, trifluoperazine, haloperidol, perphenazine, chlorprothixene, thioxine, bromperidol, loxapine and molindone.

The term “serious psychotic mental illness” denotes conditions in which delusions and/or hallucination and other manifestations of deficits in reality testing are present in conjunction with negative symptomatology. See Diagnostic and Statistical Manual,4^{th} Edition Text Revision, 2000 (DSM-IV), American Psychiatric Association, Committee on Nomenclasture and Statistics (Washington, D.C., American).

Typical symptoms associated with serious psychotic mental illnesses, include, interalia, the sudden or insidious onset of delusions or hallucinations, disorganized sppech (e.g., frequent derailment or incoherence), grossly disorganized or catatonic behavior, paranoia, and major manic, depressive or mixed episodes. Illustrative of serious psychotic mental illnesses are schizophrenia, schizoaffective illnesses, brief psychotic disorders which involve the sudden onset of delusions or hallucinations, disorganized speech or grossly disorganized or catatonic behavior. Individuals experiencing brief psychotic disorders typically experience emotional turmoil or overwhelming confusion, may have rapid shifts from one intense affect to another, and typically exhibit poor judgment, cognitive impairment or actions based on delusions.

Within the context of the present invention, “serious psychotic mental illness” also includes Bipolar I disorders typically manifested by individuals who have previously experienced at least one manic episode or mixed episode and who later manifest a manic, hypomanic, mixed or a major depressive episode which cause clinically significant distress or impairment in social, occupational or other important areas of functioning. The mood symptoms of Bipolar I disorders are not due to the direct physiological affects of substance abuse or medication and are not better accounted for by schizoaffective disorder and are not superimposed on schizophrenia, schizophreniform disorder, delusional disorder or psychotic disorder not otherwise specified.

Serious psychotic mental illness also pertains to psychotic disturbances related to specific general medical conditions and to dementias including dementia of the Alzheimer's Type and Parkinson's disease. These behavioral disturbances may take the form of hallucinations, delusions, grossly disorganized and aggressive behavior. There may be multiple medical causes or multiple types of dementia present.

In sum, serious psychotic mental illnesses include, inter alia, Schizophreniform Disorder, Schizoaffective Disorder, Severe Schizoaffective Disorder with psychotic features, Bipolar I disorders with a Single Manic Episode, Severe Bipolar I disorders with Psychotic Features, Bipolar I Disorders Manifesting a Mixed Most Recent Episode, Severe Bipolar I Disorders with Psychotic Features, Brief Psychotic Disorders, Psychotic Disorders NOS, Paranoid Personality Disorders, Schizoid Personality Disorders, Schizophrenia, Schizotypal Personality Disorders with Sedative, Hypnotic, or Anxiolytic Manifestations, Major Depressive Disorders with Psychotic Features and Psychotic Disorders due to Specific General Medical Conditions.

In a preferred embodiment, the serious psychotic disorder treated by the methods of this invention is child or early adolescent schizophrenia, in particular treatment of patient of about age 9 years to 15 years, and/or wherein the serious psychotic disorder is childhood onset schizophrenia. Childhood or early adolescent onset schizophrenia typically refers to schizophrenia showing onset from about age 9 to about age 15 years.

Preferably, the α₂ adrenergic receptor antagonist utilized according to the invention is a selective antagonist, i.e., a drug whose principal pharmacological effect in vitro is antagonism of α₂ adrenergic receptors without direct pharmacological effects on serotonin or dopamine receptors. A particularly preferred α₂ adrenergic receptor antagonist for use according the invention is idazoxan [±-2-(1,4-benzodioxan-2-yl)-d-imidazoline]. Methods of making benzodioxan imidazolines including idazoxan are disclosed in U.S. Pat. No. 2,979,511 (Krapcho et al.). Methods of formulating pharmaceutical compositions comprising benzodioxan imidazolines including idazoxan are disclosed in U.S. Pat. No. 4,436,914 (Kluge et al.) Idazoxan is a highly selective α₂ receptor antagonist. (See Doxey et al 1983). Other useful α₂ adrenergic receptor antagonists include ethoxy-idazoxan, yohimbine, fluperoxan, and atipamezole.

In a preferred embodiment of the methods of the invention, the α₂ adrenergic receptor antagonist is administered to a patient undergoing chronic treatment with an “atypical” antipsychotic. This permits assessment of the degree antipsychotic response prior to the administration of the α₂ adrenergic receptor antagonist. Thus the present invention provides a method for treating a serious psychotic mental illness in a patient in need thereof which comprises co-administration of (i) a compound having combined D₂ dopamine and 5HT₂ serotonin antagonist activities, wherein said compound has a greater antagonist affinity for D₂ dopamine receptor than its antagonist affinity for α₂ adrenergic receptor, and (ii) a compound having α₂ adrenergic receptor antagonist activity, wherein said compound (i) is administered initially alone in an amount and for a period of time sufficient to stabilize said patient and subsequently said compound (ii) is co-administered in an amount and for a period of time that allows for a reduction in the amount of compound (i) administered to said patient. The term “stabilize” (in its various grammatical forms) is used herein in the same manner recognized by those of ordinary skill, such as to represent the phase of treatment during which medication is administered at a dose that produces some clinical improvement without the occurrence of limiting adverse events. Determination of the appropriate amounts of compound administered and the duration of treatment to achieve stabilization will vary depending on illness being treated and particular patient and is within the ordinary skill of the art. In the practice of this aspect of the invention, the method may further comprise the step of reducing the amount of compound (i) administered to said patient after commencing co-administration of compound (ii), preferably reduced to approximately 50% of the dose administered to stabilize said patient. In a separate embodiment, the two compounds can be administered together at the beginning of treatment if desired.

In the practice of any of the methods of the invention one or both of the atypical antipsychotic and the α₂ antagonist can be administered as a mixture of enantiomers of said compounds or substantially in the form of a single (+) or (−) enantiomer, when the compound is susceptible to formation of separate enantiomers, i.e., which underlying compound is capable of being produced as a racemate. As used herein, the term “substantially in the form of a single enantiomer” refers to mixtures of enantiomers that comprise greater than 95/5 up to 100/0 mole ratios of either enantiomer of a racemate. In one embodiment of the methods, the atypical antipsychotic is administered in the form of a mixture of enantiomers which comprises from 95/5 to 5/95 mole ratios of the enantiomers of the particular atypical antipsychotic. In a separate embodiment of the methods the α₂ antagonist mixture comprises from 95/5 to 5/95 mole ratios of the enantiomers of the particular α₂ antagonist compound. For example, these mixtures may comprise any greater molar concentrations of one enantiomer, e.g., up to 95/5, up to 90/10, up to 80/20, up to 70/30, and up to 60/40. Preferably, the atypical antipsychotic and/or α₂ receptor antagonist will comprise the isomeric form which exhibits the best pharmacokinetic properties and/or efficacy.

In the case of idozoxan, a preferred α₂-adrenoreceptor antagonist compound according to the invention, properties of the two enantiomers of idazoxan have been compared at the pre- and post-synaptic level. (Martire, et al. 1988). Particularly, the antagonism of the two idazoxan stereoisomers was assessed at presynaptic level, by comparing their ability to antagonize clonidine at the alpha 2 adrenoreceptors regulating noradrenaline release. The antagonist (+)-idazoxan was shown to possess an affinity towards the α₂ autoreceptors 40 times higher than that of (−) idazoxan. Binding studies also revealed that (+) idazoxan was 7-8 times more potent in inhibiting the p-[³H]aminoclonidine binding. These results indicate a different affinity of α₂ adrenoreceptors for the two idazoxan steroreceptors and suggest that the α₂ adrenoreceptors located pre and post synaptically may be of different subtypes.

While in such instances, e.g., in the case of idazoxan, one isomer may be preferred, in some cases there may be no difference between the pharmacological properties of different isomers. In the case of idazoxan, the (+) stereoisomer will preferably be used, or a mixture predominantly comprising the (+) isomer, i.e., at least 80/20 ratio of (+)-, more preferably at least 90/10 ratio of +1 isomer, and most preferably at least 95/5 ratio of +1 isomer. This invention further includes administration to a patient of mixtures of either single enantiomer of a compound, or substantially in the form of either single enantiomer, according to preferred embodiments of the invention, that may nonetheless result in in vivo conversion to racemic or substantially 50/50 mole ratio mixtures of enantiomers, depending on the compound administered.

Preferred dosages of the α₂-adrenergic receptor antagonist according to the invention range from about 60 to 120 mg/day. Treatment with atypical antipsychotics should be within the recommended dose range prior to the administration of α₂ adrenergic antagonist. Once the desired dose of the α₂ adrenergic antagonist has been reached, the preferred dose of the atypical antipsychotic should be roughly 50% of the normal recommended dose range. In accordance, a 50% reduction in dose ranges for atypical antipsychotics may result in administration of compounds as follows: olanzapine, 5-12 mg/day; risperidone, 2-6 mg/day; Quetiapine, 150-450 mg/day; sertindole 2-12/mg/day; ziprasidone, 40-80 mg/day; aripiprazole, 15-30 mg/day. The Merck Manual, the Physician's Desk Reference, and individual product prescription information provide adequate disclosure for those of ordinary skill in the art to calculate appropriate dosages, and adequate reduced dosages, of the compounds contemplated for use in the methods of the invention.

In another preferred embodiment of the method according to the invention, the dose of the atypical antipsychotic should be decreased as described above to an endpoint providing for an in vivo D₂ receptor occupancy of 60% is achieved. This level of occupancy for these atypical antipsychotic drugs would only be effective when the α₂ antagonist is co-administered. In vivo D₂ occupancy can be determined using finctional brain imaging technologies such as Positron Emission Tomography (PET) or Single Photon Emission Computerized Tomography (SPECT).

Reduction of dose and D₂ occupancy for atypical antipsychotics enabled by the co-administration of α₂ antagonists would reduce risk of dose-dependent adverse events including EPS, weight gain and diabetes.

In one embodiment of the present invention, both the α₂-adrenergic receptor antagonist and the atypical antipsychotic can be administered in separate form. The two compounds can also be administered in a single pharmaceutical composition, in combination with known pharmaceutically acceptable carriers. Such pharmaceutical compositions thus constitute another aspect of the present invention. These compositions may be prepared from conventional materials by known procedures.

Further, the invention also provides a method for treating a serious psychotic mental illness comprising the step of administered to a patient in need of such treatment a therapeutically effective amount of a combination of (i) a compound that blocks or down-regulates D₂ dopamine and 5HT₂ serotonin receptor activities and (ii) a compound that blocks or down-regulates α₂ adrenergic receptor activity.

Examples of compounds that block or down-regulate D₂ dopamine and 5HT₂ serotonin receptor activities include, but are not limited to, compounds such as olanzapine, risperidone, sertindole, aripiprazole and ziprasidone. Examples of compounds that block or down-regulate α₂ adrenergic receptor activity include, but are not limited to, α₂ antagonists, norepinepherine reuptake inhibitors, selective serotonin reuptake inhibitors, and anti-sense DNA or RNA molecules and techniques.

As stated above, clozapine is the most effective of all known antipsychotics in reducing positive and negative symptoms of psychosis in otherwise treatment resistant patterns. The explanation of the enhanced efficacy of clozapine remains uncertain. However, it is known that clozapine, among its pharmacological properties, possesses a relatively equal or greater balance of affinities for α₂ and D₂ receptors. Particularly, in one report the kᵢ for clozapine for the D₂ receptor is 180 nmol whereas kᵢ for clozapine for the alpha₂ receptor is about 160 nmol (Goodman and Gilman, Pharmacol. Basis of Therapeutics, 10^{th} Edition, p. 495 (2001)); in another report the kᵢ of clozapine for the D₂ receptor is reported 130 nM and the Kᵢ for the α₂ receptor is 33 (Miyomota in Neuropsychopharmacology: The Fifth Generation of Progress, 2002, p. 778). These data translate to an estimated relative binding affinity (kᵢ) for D₂/α₂ receptors ranging from approximately 0.88 to to 4. Thus, in the practice of the present invention, the appropriate balance of D₂/α₂ receptor which may be significant to its unique therapeutic effectiveness and the beneficial properties of clozapine may be achieved during combination therapy involving the administration of at least one atypical antipsychotic and α₂ blocker.

More specifically, based thereon, it was theorized that the effectiveness of combination an atypical antipsychotic and α₂ receptor antagonist therapies for treatment of serious psychotic disorders could be improved by (i) selecting specific combinations of atypical antipsychotics and α₂ receptor antagonists such that the relative D₂/α₂ binding affinity ratios (kᵢ) range from about 0.80 to about 4.5, more preferably from about 0.85 to about 4.0, more preferably from about 0.90 to about 3.9, and most preferably ranges from about 0.95 to about 1.05, and preferably a ratio of about 1.0.

The selection of atypical antipsychotic and α₂ antagonist combinations that satisfy these binding affinity ratios will result in enhanced therapeutic efficacy of typical and atypical antipsychotics and may provide a similar relative balance of pharmacological effects to that of clozapine. This is an advantage as it will enable administration of reduced dosages thereby minimizing or eliminating adverse side effects such as tardive dyskinesia. This has particularly advantages in the context of treating children and adolescents.

Alternatively, this may be accomplished by administering the atypical antipsychotic/α₂ antagonist drug combination at dosages that are selected to offset relative differences in receptor affinities. In this regard, it is known in the art to convert antipsychotic potencies to chlorpromazine equivalents in order to compare antipsychotic dosing with different agents. Chlorpromazine equivalents are in proportion to affinities for the D₂ receptor. The target dose of clozapine is 500 mg (1000 mg chlorpromazine equivalents) for treatment-resistant patients (Merck Manual) 17^{th} Edition, (1999)). 1000 mg chlorpromazine equivalents for different D₂ blocker include 20 mg fluophenazine; 900 mg quetiapine; 24 mg sertindole; and 40 mg olanzapine.

It has been previously reported (Litman et at., Br. J Psychiatry 168:571-9, (1996)) that 1000 mg chlorpromazine equivalents of fluphenazine added to a dose of 120 mg/day of an α₂ antagonist, idazoxan, have resulted in adequate clinical responses (kᵢ for α₂ receptor for idazoxan is approximately 30 nM. However, it was not known or previously suggested that clinical responses can be provided with minimization of adverse side effects when using typical or atypical antipsychotic/α₂ antagonist drug combinations, in particular by selecting relative dosage amounts that provide for approximately the same relative occupation of D₂ and α₂ receptors. This is preferably achieved herein by selecting dosages of a antipsychotic drugs that correlates to about 900-1100 chlorpromazine equivalents, more preferably about 950-1050 chlorpromazine equivalents, and most preferably about 1000 chlorpromazine equivalents and an amount of one or more α₂ blockers that provide for equivalent occupation of D₂ and α₂ receptors. A suitable α₂ dosage can be determined by one skilled in pharmacology and will depend upon factors such as the pharmacokinetic properties of the particular α₂ drug, its α₂ receptor affinity, kᵢ, rate of absorption, half-life, ability to cross blood brain barrier, and other pharmacokinetic considerations.

Dosage equivalents for different antipsychotic drugs that correlate to 1000 mg equivalents of chlorpromazine are identified supra. Alternatively, similarly enhanced results may be achieved by combining one or more antipsychotic drugs and/or one or more α₂ antagonists so as to satisfy the above-identified dosage equivalent ratios for the antipsychotic drug and α₂ blocker. The subject improved combination therapies may be used to treat any serious psychotic illness without the adverse side effects of clozapine. In preferred embodiments, the subject combination therapies will be used to alleviate positive and negative symptoms of psychosis in otherwise treatment resistant patients, i.e., patients who have proven resistant to treatment with known typical antipsychotics alone. As mentioned, a preferred usage is for the treatment of pediatric and adolescent schizophrenia. The combination therapies of the invention further contemplate the inclusion of other drugs that enhance the synaptic effects of norepinephrine.

Drug Discovery Methods

In another embodiment of the present invention, the discovery of enhancement of antipsychotic effects by α₂ adrenergic antagonist administration to atypical antipsychotic drugs can be utilized for high throughput screening for new antipsychotic drugs. In this embodiment, drugs which show a 1-10 fold greater affinity for the α₂ receptor in comparison with affinity for the D₂ receptor, with or without antagonist effects on serotonergic receptors, are identified as candidates for new antipsychotic drugs. It is expected that high throughput screening may identify such compounds, that will then be tested in CAT and CAR animal models and may subsequently continue development for use in the clinical setting.

The present invention further provides a method for identifying compounds that are useful to treat serious psychotic mental illness which comprises subjecting a candidate compound to an assay demonstrating affinity for the D₂ dopamine receptor and an assay demonstrating affinity for the α₂ adrenergic receptor and determining that the compound demonstrates significant affinity for both the D₂ dopamine receptor and the α₂ adrenergic receptor. To refine the activities of the compounds identified, the screening method of the invention may comprise the further step of subjecting the candidate compound to an assay demonstrating affinity to 5HT₂ₐ serotonin receptor and determining that the compound does not demonstrate significant affinity for the 5HT₂ₐ or other forms of the 5HT₂ serotonin receptor. Assays for determining affinity of candidate compounds for the D₂ dopamine, α₂ adrenergic, and 5HT₂ₐ serotonin receptors are readily available to those of ordinary skill in the art and include radioligand displacement assays, reporter gene assays and stereochemical modeling. For example, the following patents describe various screens for determining receptor activity of candidate compounds: U.S. Pat. No. 6,342,360 (D₂ dopamine receptor); U.S. Pat. No. 5,994,384 (α₂ adrenergic receptor); and U.S. Pat. No. 6,140,509 (5HT₂ₐ serotonin receptor). In a preferred embodiment, the step of determining that the candidate compound demonstrates affinity for the α₂ adrenergic receptor comprises determining that the compound demonstrates between about 2 to about 15 fold lower kᵢ for the α₂ adrenergic receptor than for D₂ receptor (kᵢ nM).

Based on the description of the invention herein, it is clear that the compounds discovered in such methods would represent improved compounds for methods of treating serious psychotic mental illness. Thus, the invention also provides a method for treating a serious psychotic mental illness comprising the step of administered to a patient in need of such treatment a therapeutically effective amount of a compound identified by the methods described above. Further, the invention provides a pharmaceutical composition for treating a serious psychotic mental illness comprising a therapeutically effective amount of a compound identified by the methods described above and a pharmaceutically acceptable carrier.

Pharmaceutical Compositions

As stated above, the present invention further provides a pharmaceutical composition comprising a combination of (i) an α₂-adrenergic receptor antagonist, (ii) an atypical antipsychotic neuroleptic which has a greater antagonist affinity for D₂ dopamine receptor than its antagonist affinity for α₂ adrenergic receptor, and (iii) a pharmaceutically acceptable carrier, wherein the amount of said ingredients (i) and (ii) is therapeutically effective against serious psychotic mental illness.

Compositions within the present invention can be adapted for oral or parenteral administration, as well as for enteral administration orally or though mucus membranes, that is intranasally, sublingually, bucally or rectally, or may be adapted for slow release formulations.

Compositions for oral administration include capsules, tablets, dispersable powders, granules, syrups, elixirs and suspensions. These compositions can contain one or more conventional adjuvants such as sweetening agents, flavoring agents, coloring agents and preserving agents.

Tablets can contain the active ingredients in a mixture with conventional pharmaceutically acceptable excipients. These include inert carriers, such as calcium carbonate, sodium carbonate, lactose, and talc; granulating disintegrating agents, such as starch, gelatin acacia; and lubricating agents, such as magnesium stearate, stearic acid and talc. Tablets may be uncoated or coated by known techniques to delay disintegration and absorption in the gastrointestinal tract thereby providing a sustained action over a longer period of time.

Capsules may contain the active ingredients alone or an admixture with an inert solid carrier, such as calcium carbonate, calcium phosphate or kaolin. Similarly, suspensions, syrups and elixirs may contain the active ingredients in mixture with any of the conventional excipients utilized in the preparation of such compositions. This includes suspending agents such as methylcellulose, tracanth and sodium alginate; wetting agents such a licthin, polyoxyethyele stearate or polypoxyethylene sorbitn monoleate; and preservatives.

The pharmaceutical combination of the invention is administered in a pharmacologically-effective amount to treat any of the conditions described above, and is based on the activity of the combination. The term “pharmacologically-effective amount” means the amount of a drug or pharmaceutical agent that will elicit the biological or medical response of a tissue, system, animal or human that is being sought by a researcher or clinician. It is an amount that is sufficient to significantly affect a positive clinical response while maintaining diminished levels of side effects. The amount of pharmaceutical combination which an α₂ antagonist with a Kᵢ of approximately 40 nM may be administered to a subject in need thereof can be in the range of 60 to 120 mg/day, or more preferably 120 mg/day., administered in single or divided doses.

Administration of the dose can be oral, topical, intravenous, subcutaneous, intramuscular, or any other acceptable systemic method. Based on the judgment of the attending clinician, the amount of drug administered and the treatment regimen used will, of course, be dependent on the age, sex and medical history of the patient being treated, the severity of the specific disease condition and the tolerance of the patient to the treatment as evidenced by local toxicity and by systemic side-effects.

In practice, the pharmaceutical combinations of the invention are administered in amounts which will be sufficient to inhibit or prevent undesired medical conditions or disease in a subject, such as a mammal, and are used in the form most suitable for such purposes. The compositions are preferably suitable for internal use and include an effective amount of a pharmacologically-active compound of the invention, alone or in combination with other active agents, with one or more pharmaceutically-acceptable carriers. The compounds are especially useful in that they have very low, if any, toxicity.

The pharmaceutical combinations of the invention can form the active ingredient of a pharmaceutical composition, and are typically administered in a mixture with suitable pharmaceutical diluents, excipients or carriers (collectively referred to herein as “carrier” materials) suitably selected with respect to the intended form of administration, that is, oral tablets, capsules, elixirs, syrups and the like. The compositions typically will include an effective amount of active compound or the pharmaceutically-acceptable salt thereof, and in addition, and may also include any carrier materials as are customarily used in the pharmaceutical sciences. Depending on the intended mode of administration, the compositions may be in solid, semi-solid or liquid dosage form, such as, for example, injectables, tablets, suppositories, pills, time-release capsules, powders, liquids, suspensions, or the like, preferably in unit dosages.

Conventional pharmaceutical compositions comprising a pharmacologically-effective amount of the pharmaceutical combinations of the invention together with pharmaceutically-acceptable carriers, adjuvants, diluents, preservatives and/or solubilizers may be used in the practice of the invention. Acceptable diluents include diluents of various buffers (e.g., arginine, Tris-HCl, acetate, phosphate) having a range of pH and ionic strength, carriers (e.g., human serum albumin), solubilizers (e.g., tween, polysorbate), and preservatives (e.g., benzyl alcohol). See, for example, U.S. Pat. No. 4,496,537.

Administration of the pharmaceutical combinations described herein can be via any of the accepted modes of administration for individual therapeutic agents. These methods include systemic or local administration such as oral, nasal, parenteral, transdermal, subcutaneous, or topical administration modes.

For instance, for oral administration in the form of a tablet or capsule (e.g., a gelatin capsule), the active drug component can be combined with an oral, non-toxic pharmaceutically-acceptable inert carrier such as ethanol, glycerol, water, and the like. Moreover, when desired or necessary, suitable binders, lubricants, disintegrating agents, and coloring agents can also be incorporated into the mixture. Suitable binders include starch, magnesium aluminum silicate, starch paste, gelatin, methylcellulose, sodium carboxymethylcellulose and/or polyvinylpyrrolidone, sugars, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt, and/or polyethylene glycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

The conjugates of the invention can also be administered in such oral dosage forms as timed-release and sustained-release tablets or capsules, pills, powders, granules, elixers, tinctures, suspensions, syrups, and emulsions.

Liquid, particularly injectable compositions can, for example, be prepared by dissolving, dispersing, etc. The active compound is dissolved in or mixed with a pharmaceutically-pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically-valuable substances.

The conjugates of the present invention can be administered in intravenous (e.g., bolus or infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released system, which assures that a constant level of dosage is maintained, for example as described according to U.S. Pat. No. 3,710,795.

Furthermore, the pharmaceutical combinations of the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosols, sprays and gels, wherein the amount administered would be 10-100 times the dose typically given by parenteral administration.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The conjugates of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine, or phosphatidylcholines. In some embodiments, a film of lipid components is hydrated with an aqueous solution of drug to a form lipid layer encapsulating the drug, as described in U.S. Pat. No. 5,262,564.

The pharmaceutical combinations of the present invention may also be coupled with soluble polymers as targetable drug carriers. Such polymers can include polyalkylene glycols such as polyethylene glycol and polyethylene glycol derivatives, polyvinylpyrrolidone, pyran copolymer, polyhydroxypropyl-methacrylamide-phenol, polyhydroxyethylaspanamidephenol, or polyethyleneoxidepolylysine substituted with palmitoyl residues. The conjugates can also be coupled to proteins, such as, for example, receptor proteins and albumin. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the pharmaceutical combinations of the present invention is selected in accordance with a variety of factors including type, species, age, weight, sex and medical condition of the patient; the severity of the condition to be treated; the route of administration; the renal and hepatic function of the patient; and the particular compound or salt thereof employed. The activity of the compounds of the invention and sensitivity of the patient to side effects are also considered. An ordinarily skilled physician or veterinarian can readily determine and prescribe the effective amount of the drug required to prevent, counter or arrest the progress of the condition.

For any route of administration, divided or single doses may be used. For example, compounds of the present invention may be administered daily or weekly, in a single dose, or the total dosage may be administered in divided doses of two, three or four.

Any of the above pharmaceutical compositions may contain 0.1-99%, 1-70%, or, preferably, 1-50% of the active compounds of the invention as active ingredients.

As described above, the course of the disease and its response to drug treatments may be followed by clinical examination and laboratory findings. The effectiveness of the therapy of the invention is determined by the extent to which the previously described signs and symptoms of a condition are eliminated or substantially reduced.

The present invention is described further by reference to the following, illustrative examples.

### EXAMPLE 1

### Administration of Idazoxan in Combination with Olanzapine in Rat Models of Antipsychotic Mechanism of Action

Animals

Adult, male Wistar rats, 200-225 g (B & K Universal, Sollentuna, Sweden) were used. The animals were housed under standard laboratory conditions, on a reversed light/dark cycle (lights off 08:00 am), and allowed 1 week of adaptation to laboratory conditions before being used in experiments. Food and water were available ad libitum.

Drugs

The α₂ adrenergic receptor antagonist idazoxan and the atypical antipsychotic olanzapine were used. Idazoxan was dissolved in physiological saline and olanzapine were dissolved in a minimal (10-20 μl) amount of glacial acetic acid and made up to volume with 5.5% glucose. Idazoxan was given subcutaneously (s.c.), and olanzapine intraperitoneally (i.p.) in a volume of 2 ml/kg body weight.

Conditioned Avoidance Response Behavior (CAR)

Rats were trained and tested in a conventional, manually operated two-way active avoidance (shuttlebox) apparatus connected to a high resistance power supply. The box was divided into two compartments of equal size by a partition with one opening. Upon presentation of the 80 dB white noise conditioned stimulus (CS), the animals had 10 seconds to move from one compartment of the shuttlebox into the other. If the rat remained in the same compartment for more than 10 seconds, an intermittent electric shock (approx. 0.2 mA of 0.5-sec duration; intershock interval 2.5 sec), the unconditioned stimulus (UCS), was presented in the grid floor until an escape was performed. If the animal did not respond within 50 seconds of the shock period, the trial was terminated (escape failure). Intertrial intervals varied at random between 20 and 40 seconds. The following variables were recorded: avoidance (response to CS within 10 sec); escape (response to CS+UCS); escape failures (failure to respond); and intertrial crosses. The animals were trained for 3 consecutive days. Training sessions were run over 15 minutes. A final test session (7.5 minutes) was performed on the day immediately prior to the first experimental day, and only animals performing at the 100% correct level were assigned to the experiment. Experimental manipulations were always preceded by a pretest. All pretest and experimental sessions were run for 7.5 minutes (for further details, see e.g. Wadenberg et al. 1990). The same animals were tested repeatedly according to a change-over design (Li 1964) serving as their own controls.

Catalepsy Measurements

Animals were observed on an inclined (60°) grid. To establish a reliable baseline, the first 30 s were excluded from the actual rating time. The time the rat remained in the same position was then measured for a maximum of 2.5 min. The catalepsy was scored from 0-5 according to the time (square root transformation) the animal remained immobile (min): 0=0-0.08, 1=0.09-0.35, 2=0.36-0.80, 3=0.81-1.42, 4 =1.43-2.24, 5=≧2.25 min, i.e., if the rat remained immobile for >2.25 min it was scored as 5, etc (see Ahlenius and Hillegaart 1986).

Statistics

Statistical analysis was performed by means of the Friedman two-way ANOVA by ranks, followed by the Wilcoxon matched-pairs signed-ranks test for comparisons with control conditions (CAR), or the the Kruskal-Wallis one-way ANOVA by ranks followed by the Mann-Whitney U-test for comparisons with vehicle treated controls (catalepsy) (Siegel and Castellan Jr 1988).

Results

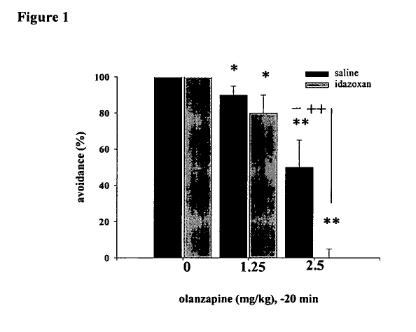

FIG. 1 and Table 1 present the experimental results for this example.

TABLE 1 shows the effects of olanzapine (2.5 mg/kg i.p.) and idazoxan (1.5 mg/kg s.c.), alone or in combination, on catalepsy in rats. Shown are CAT ratings±semi-interquartile range based on 8 animals per treatment group. Olanzapine and idazoxan did not produce any significant catalepsy compared to vehicle treated controls either alone or in combination.

TABLE 1
Time after olanzapine 2.5 mg/kg i.p. (hrs)

| Drug | 0.5 | 1 | 2 |
|---|---|---|---|
| veh/veh | 0 ± 0.5 | 1.0 ± 0.5 | 0.5 ± 0.5 |
| ida/veh | 0 ± 0.5 | 0.5 ± 0.5 | 0 ± 0.3 |
| Veh/ozp | 1 ± 2.0 | 1.5 ± 2.0 | 1 ± 0.5 |
| Ida/ozp | 1 ± 0 | 0.5 ± 0.5 | 0.5 ± 0.5 |

The present series of experiment has used the preclinical CAR and CAT tests with high predictive validity for antipsychotic activity and extrapyramidal side effect (EPS) liability, respectively. It was discovered in rats that adjunctive treatment with a selective alpha2 adrenergic receptor antagonist to relatively low doses of a commonly used antipsychotics with low affinity for α₂ adrenergic receptors produced a significant antipsychotic-like effect without catalepsy. Previous data (Wadenberg et al., 2001) have demonstrated that an effective suppression of CAR, i.e. 80-100%, requires about 5 mg/kg of olanzapine in the rat. The present results, obtained by a combination of idazoxan and olanzapine, demonstrate an equally or more effective suppression of the CAR by the use of only 2.5 mg/kg of olanzapine. Thus, these data indicate that the dose of olanzapine required to obtain an effective antipsychotic effect might be reduced by almost 50% through the adjunct treatment with idazoxan. Our results generally predict that if a selective alpha2 adrenergic receptor antagonist is given as an adjunct, it may be possible to achieve sufficient antipsychotic effect using a lower dose of an antipsychotic drug than is normally needed when the antipsychotic drug is given alone. Since lower doses of the antipsychotics will generate decreased D2 occupancy, a safer EPS liability profile should be created by the drug combination, as indicated by the present catalepsy results.

### EXAMPLE 2

### Formulations

Pharmaceutical compositions according to the present invention can include the α₂-adrenergic receptor antagonist and the atypical antipsychotic in various proportions. For example, a tablet can include olanzapine and idazoxan in the proportions of 5 mg: 40-80 mg. A capsule can include risperidone and idazoxan in the proportions of 1.5 mg: 40-80 mg.

### EXAMPLE 3

### Screening for New Drug Candidates

Prospective pharmaceutical agents useful for the treatment of serious mental illness according to the present invention can be discovered using a receptor affinity (Kᵢ) profile proportions for the α₂ and D₂ receptors in the following proportions: 20 nM: 40 nM; 20 nM: 100 nM; 15 nM: 150 nM.

### EXAMPLE 4

### Administration of Quetiapine in Combination with Idazoxan

Six patients (three males, three females) who meet DSM-III R criteria for schizophrenia and who have no medical or neurological illness and give informed consent for a pharmacological study during which quetiapine is administered in combination with idazoxan with dosage amounts approximating 1000 mg equivalents of chlorpromazine and 120 mg of idazoxan. This translates to a dosage of quetiapine of 900 mg and a dosage of idazoxan of 120 mg. These patients are given capsules containing 900 mg of quetiapine and “blinded” formulation of 120 mg of idazoxan daily for 4 to 6 weeks. Another similar group of patients is treated with placebo idazoxan capsules.

Additionally, another group of patients is treated with quetiapine and blinded idazoxan at dosages that do not fall within the preferred dosage ranges of the present invention, i.e., 300 mg or 1500 mg of quetiapine and 120 mg idazoxan.

These patients are then evaluated by physicians who are blind to whether the patient is administered the active drug combination or capsules. This evaluation is effected by the Brief Psychiatric Rating Scale, defined in Overall et al., Psychol. Rep. 10: 799-812 (1961) and the Simpson Neurological Rating Scale, defined in Simpson et al., Acta Psychiatr. Scand. 212 (Supp.):9-I 1 (1970) for drug side effects weekly.

Additionally, blood samples are taken to monitor drug plasma levels. Rating Scale total scores are averaged for each group and compared to confirm that the group administered the atypical antipsychotic α₂ antagonist combination in the preferred D₂/α₂ ratio exhibits enhanced rating score reduction relative to the placebo idazoxan group and the group given the same drugs but in dosage ratios not falling within preferred D₂/α₂ ratios

### REFERENCES

Ahlenius S, Hillegaart V (1986) Involvement of extrapyramidal motor mechanisms in the suppression of locomotor activity by antipsychotic drugs: a comparison between the effects produced by pre- and postsynaptic inhibition of dopaminergic neurotransmission. Pharmacol Biochem Behav 24: 1409-1415
Arnt J, Skarsfeldt T. Do novel antipsychotics have similar pharmacological characteristics? A review of evidence. Neuropsychopharmacology 1998; 18-63-101.
Arnt J. Pharmacology specificity of conditioned avoidance response inhibition in rats: Inhibition by antipsychotics and correlation to dopamine receptor blockage. Acta Pharmacol Toxicol 1982;51:321-329.
Baldessarini R J and Tarazi F I. Drugs and the Treatment of Psychiatric Disorders: Psychosis and Mania, in Goodman & Gilman's The Pharmacological Basis of Therapeutics 10^{th} Edition, 2001, pp 485-520.
Chakos M, Lieberman J, Hoffman E, Bradford D, Brain Sheitman. Effectiveness of second-generation antipsychotics in patients with treatment-resistant schizophrenia: A review and meta-analysis of randomized trials. Am J Psychiatry 2001; 158:518-526
Creese I, Burt D R, Snyder S H. Dopamine receptor binding predicts clinical and pharmacologic potencies of antischizophrenic drugs. Science 1976; 192:481-483
Diagnostic and Statistical Manual,4^{th} Edition Text Revision, 2000 (DSM-IV), American Psychiatric Association, Committee on Nomenclasture and Statistics, Washington, D.C.
Doxey J C, Roach A G, Smith C F. Studies on RX781094: a selective, potent and specific antagonist of alpha 2-adrenoceptors. British Journal of Pharmacology 1983. 78:489-505.
Farde L, Nordstrom A L, Wiessel F A, Pauli S, Halldin C, Sedvall G. Positron emission tomographic analysis of central D1 and D2 dopamine receptor occupancy in patients treated with classical antipsychotics and clozapine: Relation to extrapyramidal side effects. Arch Gen Psychiatry 1992;49:538-544.
Haan L, van Bruggen M Lavalaye J, Booij J et al. Subjective experience and D₂ receptor occupancy in patients with recent-onset schizophrenia treated with low-dose olanzapine or haloperidol: A randomized, double-blind study. Am J Psychiatry 2003 160:303-309.
Hertel P, Fagerquist M V, Svensson T H. Enhanced Cortical Dopamine Output and Antipsychotic-like Effects of Raclopride by α₂ Adrenoceptor Blockade, Science 1999 286:105-107.
Kane J, Honigfeld G, Singer J, Meltzer H. Clozapine for the treatment-resistant schizophrenic. A double-blind comparison with chlorpromazine. Arch Gen Psychiatry 1988;45:789-796.
Kapur S, Zipursky R, Remington G. Clinical and theoretical implications of 5-HT2 and D2 receptor occupancy of clozapine, risperidone, and olanzapine in schizophrenia. Am J Psychiatry 1999 156:286-293.
Kapur S, Seeman P. Does fast dissociation from the dopamine D₂ receptor explain the action of atypical antipsychotics? A new hypothesis. Am J Psychaitry 2001 158:360-369.
Kapur S, Zipursky R, Jones, C et al. Relationship between dopamine D(2) occupancy, clinical response and side effects: A double-blind PET study of first episode schizophrenia. Am J Psyhciatry 2000 157:514-520.
Li C C (1964) Introduction to experimental statistics. McGraw-Hill, N.Y., pp. 207-226 Siegel S, Castellan Jr N J (1988) Nonparametric Statistics for The Behavioral Sciences. McGraw-Hill, New York. Litman R E, Su T-P, Potter W Z, Hong W W, Pickar D: Idazoxan and response to typical antipsychotics in treatment-resistant schizophrenia: comparison with the atypical antipsychotic, clozapine. Br J Psychiatry 1996;168:571-579.
Martire, M, Pistritto, G, Preziosi, P. α₂-Adrenoceptor blocking properties of idazoxan stereoisomers: stereoselectivity for presynaptic α₂-adrenoceptors. Neuroscience Letters, 1988 86:328-333.
Meltzer H Y, Matsubara S, Le J C. Classification of typical and atypical antipsychotic drugs on the basis of dopamine D₁, D₂, and serotonin₂pKi values. J Pharmacol Exp Ther 1989 251:238-246.
Meltzer H V, Mechanism of Action Of Atypical Antipsychotic Drugs in Neuropsychopharmacology: The Fifth Generation of Progress, Davis K L, Charney D C, Coyle J T, Nemeroff C (Eds), 2001, Lippincott Williams & Wilkins, Philadelphia, pp. 819-831.
Meltzer H Y, Alphs L, Green A I, Altamura A C, Anand R, Bertoldi A, Bourgeois M, Chouinard G, Islam M Z, Kane J, Krishnan R, Lindenmayer J P, Potkin S; International Suicide Prevention Trial Study Group. Clozapine treatment for suicidality in schizophrenia: International Suicide Prevention Trial (InterSePT). Arch Gen Psychiatry. 2003 60(1):82-91.
Miyamoto S, Duncan G E, Goff D C, et al. Therapeutics of Schizophrenia in Neuropsychopharmacology: The Fifth Generation of Progress, Davis K L, Charney D C, Coyle J T, Nemeroff C (Eds), 2001, Lippincott Williams & Wilkins, Philadelphia, pp. 775-807.
Nyberg S, Farde L, Eriksson L, et al. D₂ dopamine receptor occupancy in the living human brain: a PET study with risperidone. Psychopharmacology (Berl) 1993: 110 265-272.
Nordstrom Al, Nyberg S, Olsson H, Farde L: Positron emission tomography finding of a high striatal D-2 receptor occupancy in olanzapine-treated patients. Arch Gen Psychiatry 1998;55:283-284.
Pickar D, Owen R R, Litman R E, Konicki P E, Gutierrez R, Rapaport H M: Clinical and biological response to clozapine in patients with schizophrenia: crossover comparison with fluphenazine. Arch Gen Psychiatry 1992;49:345-353.
Pickar D: Prospects for the pharmacotherapy of Schizophrenia. The Lancet 1995;345:557-562.
Pickar D, Su T-P, Weinberger D, Coppola R, Malhotra A K, Knable M B, Lee K S, Gorey J, Bartko J J, Breier A, Hsiao J: Individual variation in D₂ dopamine receptor occupancy in clozapine-treated patients. Am J Psychiatry 1996;153:1571-1578.
Pickar D, Bartko J J. Effect size of drug withdrawal and subsequent clozapine administration in “Treatment Resistant” schizophrenic patients. Am J Psychiatry 2003, 160:113-1138.
Seeman P, Lee T, Chau-Wong M, Wong K. Antipsychtoic drug doses and antipsychotic/dopamine receptors. Nature 1976;261:717-719.
Shapiro D A, Renock S, Arrington E, et al. Aripiprazole, a novel atypical antipsychotic drug with a unique and robust pharmacology. Neuropsychopharmacology 2003, online publication
Snyder S H. The dopamine hypothesis of schizophrenia: focus on the dopamine receptor. Am J Psychiatry 1976;133:197-202.
Su T P, Malhotra A K, Hadd K, Breier A, Pickar D. D₂ dopamine receptor occupancy: Crossover comparison of risperidone with clozapine therapy in schizophrenic patients. Archives of General Psychiatry, 1997;54:972-3.
Wadenberg M-L, Ericsson E-L, Magnusson O and Ahlenius S (1990) suppression of conditioned avoidance behavior by the local application of (−)sulpiride into the ventral, but not the dorsal, striatum of the rat. Biol Psychiatry 28:297-307.
Wadenberg, J-L G, Soliman A, Vanderspek, et al. Dopamine D₂ receptor occupancy is a common mechanism underlying animal models of antipsychotics and their clinical effects. Neuropsychopharmacology 25:633-641, 2001.
Wadenberg M-L G, Kapur S, Soliman A, et al. Dopamine D₂ receptor occupancy predicts catalepsy and the suppression of conditioned avoidance response behavior in rats. Psychopharmacology (Berl) 150:422-429.

Many modifications and variations of this invention can be made without departing from its spirit and scope, as will be apparent to those skilled in the art. The specific embodiments described herein are offered by way of example only, and the invention is to be limited only by the terms of the following claims, along with the full scope of equivalents to which such claims are entitled.

What is claimed is:

1. A method for treating a serious psychotic mental illness comprising the step of administering to a patient in need of such treatment a therapeutically effective amount of a combination of (i) an α₂ -adrenergic receptor antagonist and (ii) an atypical antipsychotic neuroleptic which has a greater antagonist affinity for D₂ dopamine receptor than its antagonist affinity for α₂ adrenergic receptor in a pharmaceutically acceptable carrier.
